(19) United States
(12) Patent Application Publication
(10) Pub. No.: US 2015/0173892 A1
(43) Pub. Date: Jun. 25, 2015

(54) ACCOMMODATING INTRAOCULAR LENS

(71) Applicant: NOVARTIS AG, BASEL (CH)

(72) Inventors: DAVID BORJA, FORT WORTH, TX (US); LAUREN DeVITA GERARDI, DE PLAINES, IL (US)

(21) Appl. No.: 14/524,681

(22) Filed: Oct. 27, 2014

Related U.S. Application Data
(60) Provisional application No. 61/919,897, filed on Dec. 23, 2013.

(51) Int. Cl. A61F 2/16

(52) U.S. Cl. CPC A61F 2/1635; A61F 2002/1683

(57) ABSTRACT

An accommodating intraocular lens includes a haptic assembly and a flexible optic. The haptic assembly includes an anterior ring, a posterior ring, anterior spring arms, and posterior spring arms, wherein the anterior spring arms and the posterior spring arms bias the anterior ring and the posterior ring apart from one another. The flexible optic is suspended between the anterior ring and the posterior ring and connected to the haptic assembly by a plurality of support struts. The support struts are adapted to deform the flexible optic upon axial compression of the haptic assembly so that an optical power of the flexible optic is reduced relative to an uncompressed state of the haptic assembly.

This application claims priority to U.S. Provisional Application No. 61/919897 filed Dec. 23, 2013.

### TECHNICAL FIELD

This invention relates generally to the field of accommodating intraocular lenses and, more particularly, to a haptic design for a curvature changing accommodating intraocular lens.

### BACKGROUND OF THE INVENTION

The human eye in its simplest terms functions to provide vision by transmitting light through a clear outer portion called the cornea, and focusing the image by way of a crystalline lens onto a retina. The quality of the focused image depends on many factors including the size and shape of the eye, and the transparency of the cornea and the lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, an opening is made in the anterior capsule and a thin phacoemulsification cutting tip is inserted into the diseased lens and ultrasonically vibrated. The vibrating cutting tip liquefies or emulsifies the lens so that the lens may be aspirated out of the eye. The diseased lens, once removed, is replaced by an IOL.

In the natural lens, distance and near vision is provided by a mechanism known as accommodation. The natural lens is contained within the capsular bag and is soft early in life. The bag is suspended from the ciliary muscle by the zonules. Relaxation of the ciliary muscle tightens the zonules, and stretches the capsular bag. As a result, the natural lens tends to flatten. Tightening of the ciliary muscle relaxes the tension on the zonules, allowing the capsular bag and the natural lens to assume a more rounded shape. In this way, the natural lens can focus alternatively on near and far objects.

As the lens ages, it becomes harder and is less able to change its shape in reaction to the tightening of the ciliary muscle. Furthermore, the ciliary muscle loses flexibility and range of motion. This makes it harder for the lens to focus on near objects, a medical condition known as presbyopia. Presbyopia affects nearly all adults upon reaching the age of 45 to 50. Various accommodative intraocular lenses (IDLs) have been proposed. However, due to limited residual accommodative forces, the mechanical design required to effectively translate accommodative force into changes in optical power has proved challenging.

### BRIEF DESCRIPTION OF THE DRAWINGS

FIG. 1 illustrates an accommodating intraocular lens according to an embodiment of the invention; and

FIGS. 2A and 2B illustrate a cross-sectional side view of the lens of FIG. 1, respectively, in a disaccommodated and accommodated state.

### SUMMARY OF THE INVENTION

Various embodiments of the present invention provide an accommodating intraocular lens. In a particular embodiment, an accommodating intraocular lens includes a haptic assembly and a flexible optic. The haptic assembly includes an anterior ring, a posterior ring, anterior spring arms, and posterior spring arms, wherein the anterior spring arms and the posterior spring arms bias the anterior ring and the posterior ring apart from one another. The flexible optic is suspended between the anterior ring and the posterior ring and connected to the haptic assembly by a plurality of support struts. The support struts are adapted to deform the flexible optic upon axial compression of the haptic assembly so that an optical power of the flexible optic is reduced relative to an uncompressed state of the haptic assembly.

The embodiments discussed below are exemplary, and various changes can be made to these illustrative embodiments without deviating from the scope of the invention. For example, the features of one embodiment can be combined with those of another embodiment.

### DETAILED DESCRIPTION

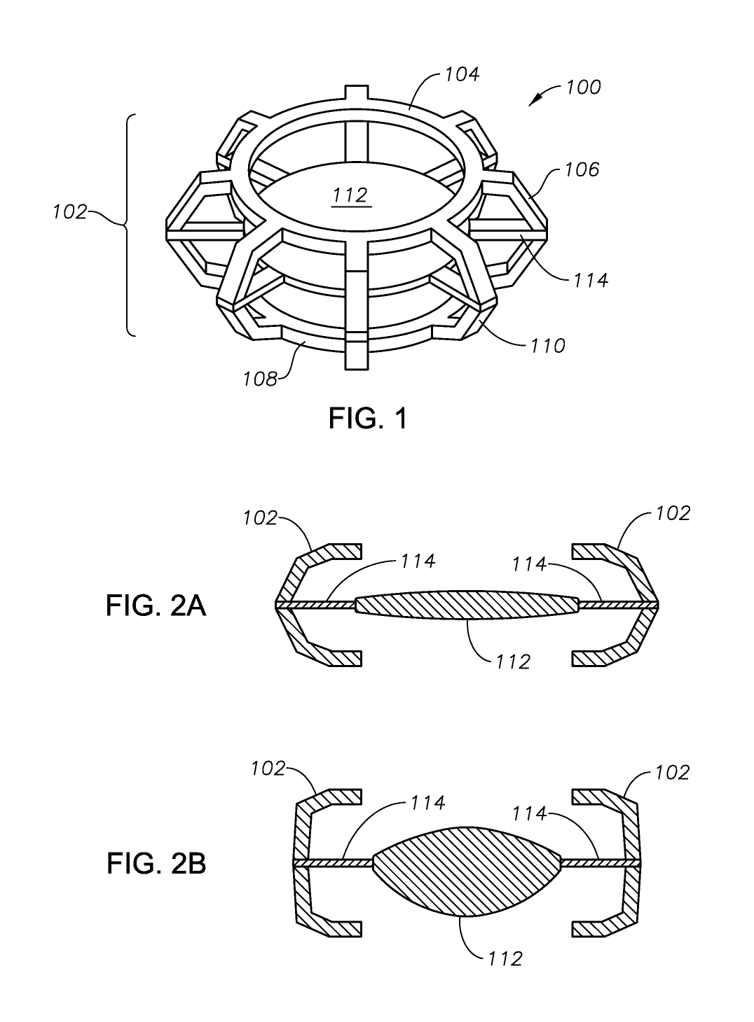

As shown in FIG. 1, an accommodating intraocular lens 100 includes a haptic assembly 102 including an anterior ring 104 with anterior spring arms 106, a posterior ring 108 with posterior spring arms 110. Transparent windows (not shown) may be placed within either or both of the anterior ring 104 and posterior ring 108. The lens 100 also includes flexible optic 112 that is connected to the haptic assembly 102 by support struts 114. The anterior spring arms 106, the posterior spring arms 110, and the support struts 114 may be equally spaced around the flexible optic 112. The haptic assembly 102 is adapted to maintain the general shape of the capsular bag when implanted therein, which helps to maintain the mechanical operation of the capsular bag. The flexible optic 112 may be any soft optic that can be stretched by tension on the support struts produced by transfer of axial force to the haptic assembly. For example, the flexible optic 112 may be a fluid- or gel-filled membrane or an elastic polymeric material.

The operation of the lens 100 is illustrated in FIGS. 2A and 2B. In operation, when the capsular bag is flattened by zonular tension in a disaccommodated state (FIG. 2A), the axial force on the haptic assembly 102 forces the anterior and posterior rings toward one another, pulling the support struts 114 outwardly and flattening the flexible optic 112. This allows the optic 112 to have reduced optical power in the disaccommodated state. When the zonular tension is relaxed during accommodation (FIG. 2B), the axial force on the haptic assembly 102 is reduced, and the spring action of the spring arms 106 and 110 forces the anterior ring 104 and the posterior ring 108 apart. This relaxes the tension in the support struts 114, allowing the flexible optic 112 to return to a more rounded state, increasing the optical power.

Those having ordinary skill in the art will appreciate that various changes can be made to the above embodiments without departing from the scope of the invention.

What is claimed is:

1. An accommodating intraocular lens, comprising:
a haptic assembly comprising an anterior ring, a posterior ring, anterior spring arms, and posterior spring arms, wherein the anterior spring arms and the posterior spring arms bias the anterior ring and the posterior ring apart from one another; and
a flexible optic suspended between the anterior ring and the posterior ring and connected to the haptic assembly by a plurality of support struts, wherein the support struts are adapted to deform the flexible optic upon axial compression of the haptic assembly so that an optical power of the flexible optic is reduced relative to an uncompressed state of the haptic assembly.

2. The lens of claim 1, wherein the lens further comprises a transparent window within at least one of the anterior ring or the posterior ring.

3. The lens of claim 1, wherein the flexible optic is a fluid-filled membrane.

4. The lens of claim 1, wherein the flexible optic is a gel-filled membrane.

5. The lens of claim 1, wherein the flexible optic is an elastic polymer.
